(19) United States
(12) Patent Application Publication
(10) Pub. No.: US 2023/0310691 A1
(43) Pub. Date: Oct. 5, 2023

(54) FLUID DISPERSAL APPARATUS FOR AIR QUALITY CONTROL OF TRANSITORY SOURCE

(71) Applicant: ABOVE ALL MISTERS LLC, Queen Creek, AZ (US)

(72) Inventor: Michael Hearns, Queen Creek, AZ (US)

(73) Assignee: ABOVE ALL MISTERS LLC, Queen Creek, AZ (US)

(21) Appl. No.: 18/128,195

(22) Filed: Mar. 29, 2023

Related U.S. Application Data
(60) Provisional application No. 63/324,300, filed on Mar. 28, 2022.

(51) Int. Cl. A61L 11/00; B05B 1/20; B05B 15/68; B05B 9/01; A61L 9/14; A61L 9/01

(52) U.S. Cl. CPC A61L 11/00; B05B 1/20; B05B 15/68; B05B 9/01; A61L 9/14; A61L 9/01; A61L 2209/21; A61L 2209/134; A61L 2209/11

(57) ABSTRACT

An apparatus for dispensing an air quality control fluid over refuse includes a movable base with a self-contained fluid reservoir, a pump, and an array of nozzles for dispensing the fluid. A method for using the apparatus includes relocating the apparatus as locations of fresh refuse or air movement over the pile shifts.

### CROSS REFERENCE TO RELATED APPLICATION

The present application claims priority to U.S. provisional patent application Ser. No. 63/324,300 filed Mar. 28, 2022, which is incorporated herein in its entirety by reference.

### FIELD

The present application relates to de-using an apparatus, system or method of dispersing an air quality control fluid over a refuse pile or other transitory source of air contaminants, for example, dust or undesired odors.

### BACKGROUND

Refuse piles and similar transitory sources of unpleasant odors and dust are a problem in urban and suburban areas, especially where development encroaches on formerly remote processing facilities. Operators may improve air quality impacts by dispersing an air quality control fluid in a fine mist over the operating area. Air quality control fluids may have one or more targeted effects, for example for odor control, dust control, chemical contaminant control, or any combination of these. Dispersed as a fine mist, the air quality control fluid reacts with the targeted contaminant, rendering it inoffensive by chemical reaction or by removing it from the air, for example by absorption and precipitation.

For example, odor control fluids may use enzymes to break down odoriferous organic compounds into odorless molecules as known in the art. Dust control fluids may include surfactants that help trap dust in the fluid particle that drops to the ground, precipitating dust out of the air. Chemical control fluids may include reactants for the targeted contaminant, that react when dissolved in the air or in the carrier fluid. Air quality control fluids may be dispensed onto or over the source of the undesired odors where they can be most effective. In other applications, contaminated air can be suctioned away for charcoal filtration, enzyme application, or other treatment.

Where the contaminant source is fixed, as in a processing plant, an air quality control system can be made part of the physical facility and customized for the processing application. In many outdoor applications, however, the contaminated air is uncontained while the source of the contamination does not have a fixed location. In refuse piles, for example, most odor and dust comes from fresh piles of refuse before the piles are covered, incinerated, or otherwise processed. In landfill applications, for example, the air contamination source moves continually as fresh refuse is deposited and older refuse is buried.

For such outdoor, non-static odor sources, air quality management often includes blowing a mist of air quality control fluid over the contaminant source, such as a fresh refuse pile. The blower and spray system are compact and easily moved around. However, is it difficult to evenly cover any area using a blower, because wind conditions and the position of the blower change frequency. Much of the air quality control fluid blows away without performing its intended function. On the other hand, a more permanent installation is not feasible because the source of odor moves frequently.

It would be desirable, therefore, to develop new methods and other new technologies for air quality control over outdoor sources that are not in a fixed location, or that frequently change depending on use and weather conditions, that overcomes these and other limitations of the prior art.

### SUMMARY

This summary and the following detailed description should be interpreted as complementary parts of an integrated disclosure, which parts may include redundant subject matter and/or supplemental subject matter. An omission in either section does not indicate priority or relative importance of any element described in the integrated application. Differences between the sections may include supplemental disclosures of alternative embodiments, additional details, or alternative descriptions of identical embodiments using different terminology, as should be apparent from the respective disclosures.

In an aspect of the disclosure, an apparatus for dispensing an air quality control fluid over refuse, the apparatus may include a base supporting a vertical mast and a boom coupled to the mast by a pivoting connector configured to allow the boom to move between a folded position against the mast and a horizontally extended position. The apparatus may further include a fluid container for holding an air quality control fluid coupled to the base; a fluid pump coupled to the container and to an output fluid line disposed for directing air quality control fluid along the boom and an array of fluid dispensing nozzles extending along a length of the boom. The fluid container may be a plastic container mounted to the base. The array of nozzles is coupled to receive the air quality control fluid from the output fluid line. The array of fluid dispensing nozzles may be oriented to emit the air quality control fluid downwards, or in any other desired direction, for example, horizontally or upwards, depending on the air quality control application.

The apparatus may further include a piston-driven actuator, for example, a hydraulic cylinder, interposed between the boom and at least one of the base or the mast, configured for moving the boom between the horizontally extended position and the folded position. The boom can be lowered and folded to facilitate relocating the apparatus as the odor source moves. The apparatus may further include a hinge that couples segments of the boom together, enabling the boom to be folded. In an aspect, the hinge is spring-loaded to assist manual folding of the boom when lowered. The boom may be, or may include, a truss.

In some embodiments, the piston driven actuator may be a hydraulic cylinder. Accordingly, the apparatus my further include a hydraulic system coupled to the hydraulic cylinder.

In another aspect, the apparatus may include at least one skid supporting the base. For example, the skid may be, or may include, a steel plate.

The apparatus may further include an electrical system coupled to the fluid pump. The electrical system may include a control panel coupled to the electrical system, an electrical port for connecting a source of electric power to the electrical system, or both. The system may include a portable generator coupled to the electrical system via the port.

In another aspect, a method for dispensing an air quality control fluid over a refuse pile may include placing an apparatus on or adjacent to a refuse pile, the apparatus including a base supporting a vertical mast, wherein a boom is coupled to the mast by a pivoting connector configured to allow the boom to move between a folded position against the mast and a horizontally extended position. The method may further include providing the air quality control fluid in a container coupled to the base. The method may include powering a fluid pump coupled to the container and to an output fluid line disposed for directing air quality control fluid along the boom. The method may include dispensing the air quality control fluid through an array of fluid dispensing nozzles extending along a length of the boom and coupled to receive the air quality control fluid from the output fluid line. The method may include folding segments of the boom to place the boom in the folded position.

To the accomplishment of the foregoing and related ends, one or more examples comprise the features hereinafter fully described and particularly pointed out in the claims. The following description and the annexed drawings set forth in detail certain illustrative aspects and are indicative of but a few of the various ways in which the principles of the examples may be employed. Other advantages and novel features will become apparent from the following detailed description when considered in conjunction with the drawings and the disclosed examples, which encompass all such aspects and their equivalents.

### BRIEF DESCRIPTION OF THE DRAWINGS

The features, nature, and advantages of the present disclosure will become more apparent from the detailed description set forth below when taken in conjunction with the drawings in which like reference characters identify like elements correspondingly throughout the specification and drawings.

FIG. 1 is a perspective view illustrating a movable apparatus for dispensing an air quality control fluid, with the boom in an extended position.

FIG. 2 is a side view further illustrating the apparatus of FIG. 1.

FIG. 3 is a front view illustrating the apparatus of FIG. 1, with the boom in a folded position.

FIG. 4 is a side view illustrating the apparatus of FIG. 3.

FIG. 5 is a perspective view illustrating the base and mast components of the apparatus, without the boom and related components.

FIG. 6 is a perspective view illustrating the base, and a lower portion of the boom in a folded position, with the door to the interior of the base closed.

FIG. 7 is a perspective view like FIG. 6, showing the door to the interior of the base open.

FIG. 8 is a perspective view illustrating interior components of the base.

FIG. 9 is a conceptual block diagram illustrating components of electrical and fluid handling components of the apparatus for dispensing an air quality control fluid.

FIGS. 10 and 11 are side and perspective views, respectively, of an alternative embodiment including dual folding booms.

FIG. 12 is a side view showing an alternative mechanism for folding a boom of the fluid dispensing apparatus.

FIG. 13 is a flow chart illustrating aspects of a method for dispensing an air quality control fluid.

### DETAILED DESCRIPTION

Various aspects are now described with reference to the drawings. In the following description, for purposes of explanation, numerous specific details are set forth to provide a thorough understanding of one or more aspects. It may be evident, however, that the various aspects may be practiced without these specific details. In other instances, well-known structures and devices are shown in block diagram form to facilitate describing these aspects.

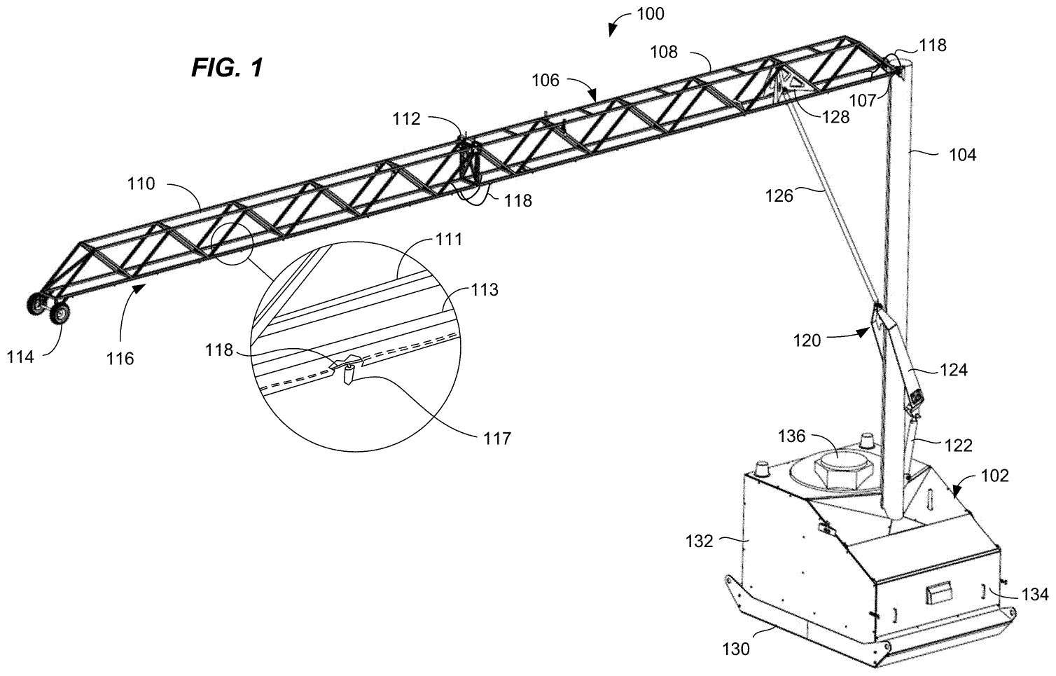

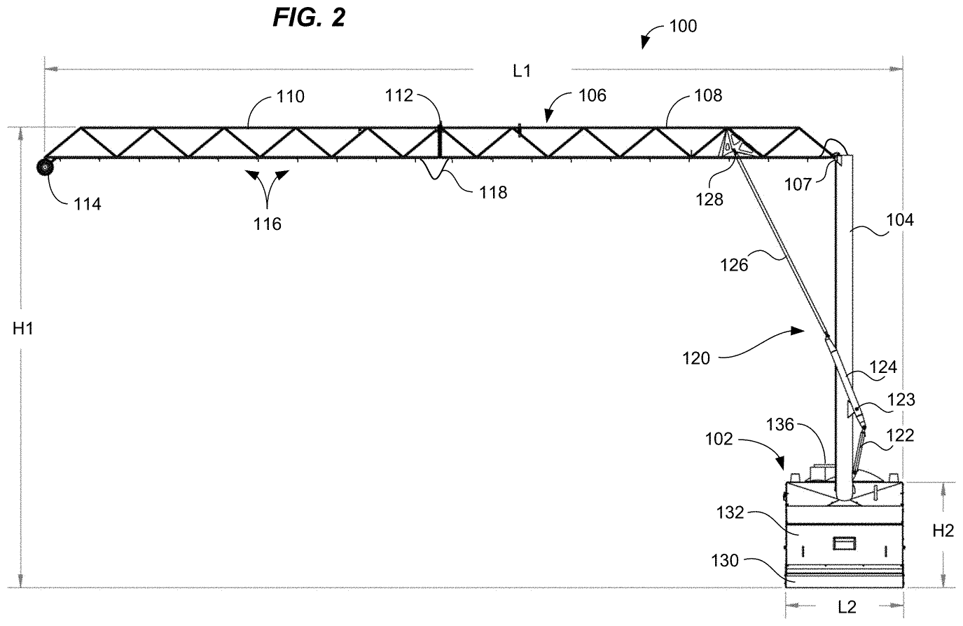

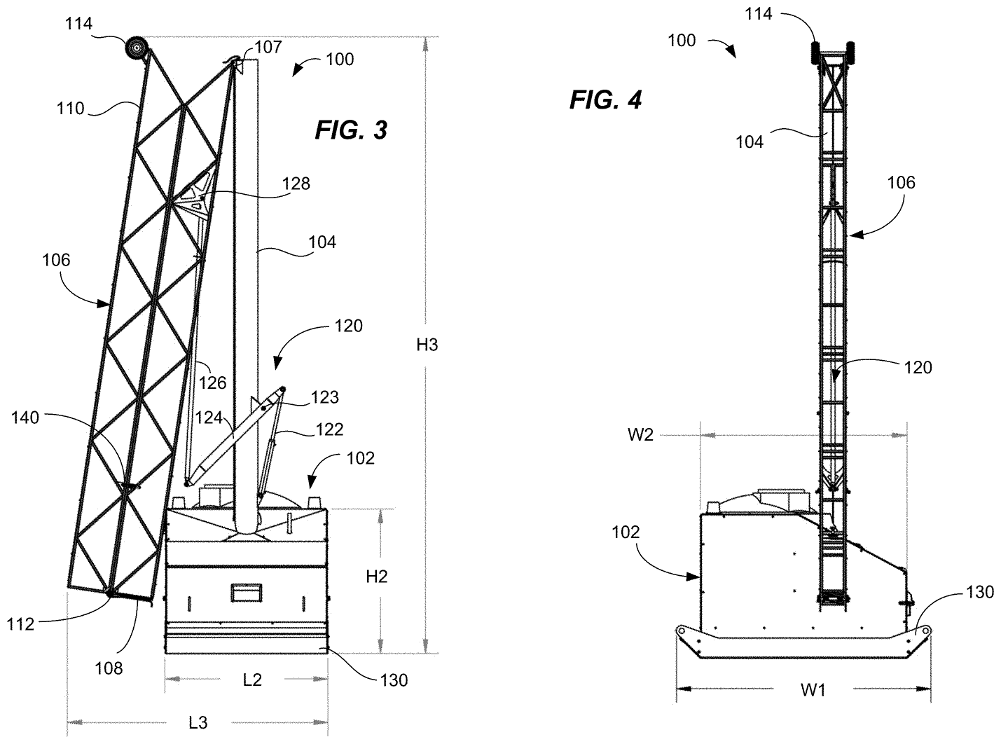

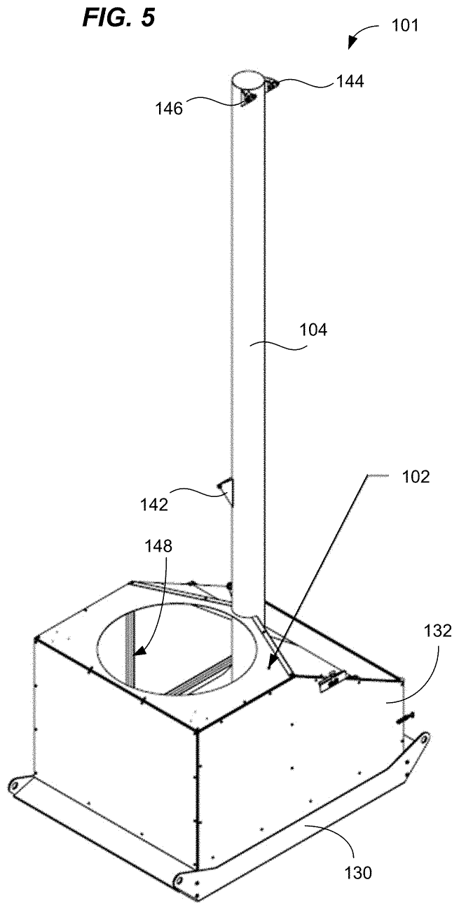

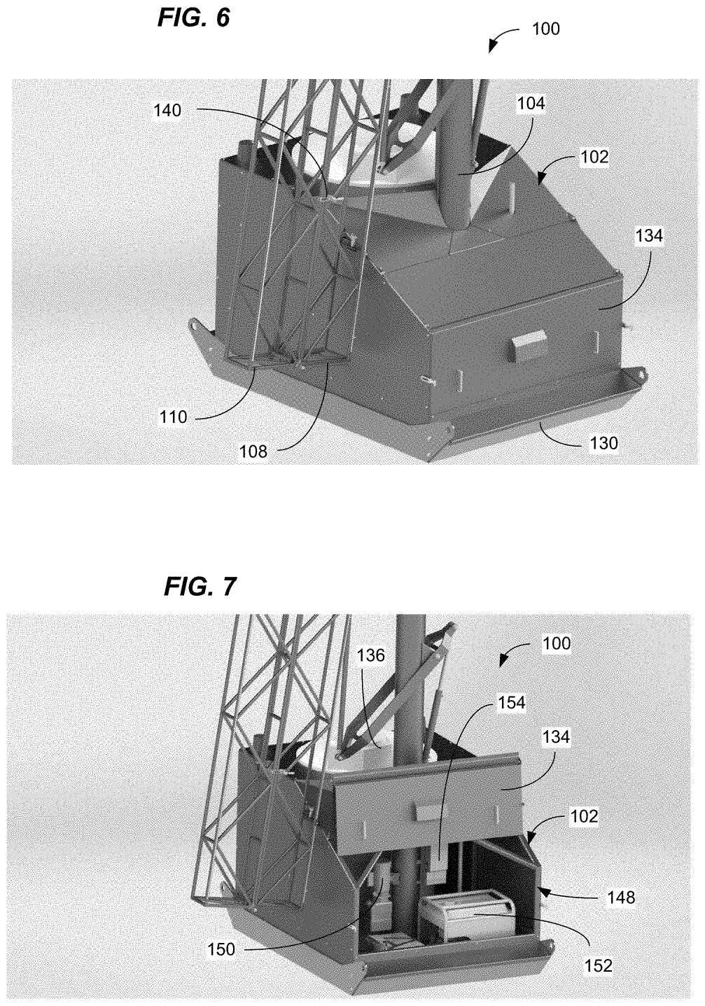

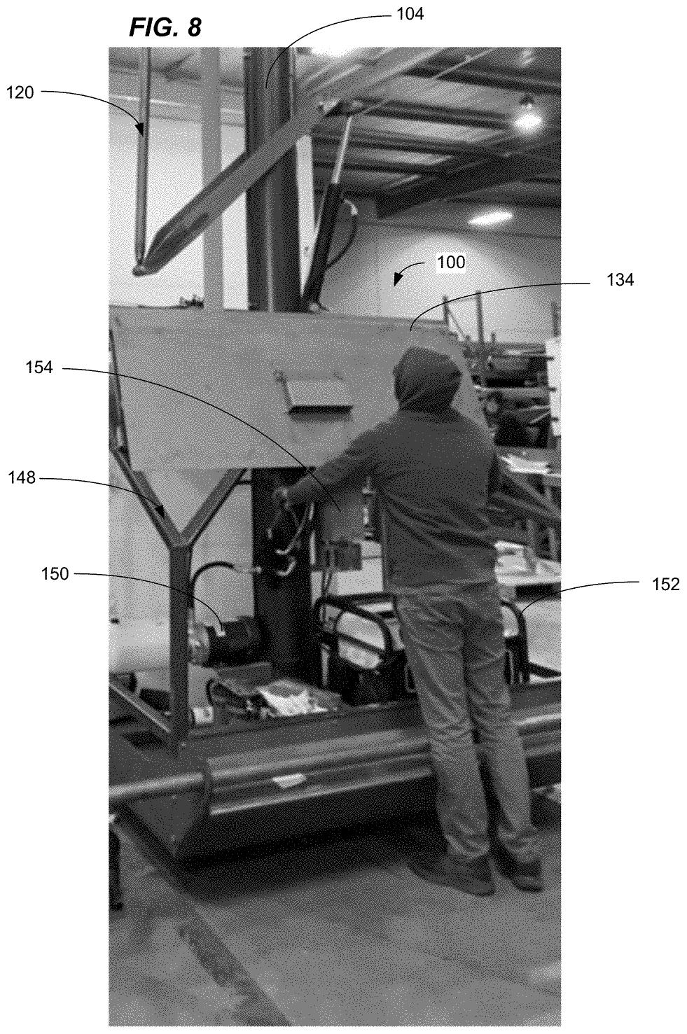

Referring to FIGS. 1-8, an apparatus 100 for dispensing an air quality control fluid over a refuse pile or other transitory odor source may include a base 102 supporting a vertical mast 104 by a frame 148 shown in FIGS. 5, 7 and 8. The apparatus may include a boom 106 coupled to the mast 104 by a pivoting connector 107 configured to allow the boom to move between a folded position against the mast as shown in FIGS. 3-4 and a horizontally extended position as shown in FIGS. 1-2. The pivoting connector 107 may be a hinge formed from the brackets 144, 146 shown in FIG. 5, corresponding pin bearings (not shown) in the end of the boom 106 proximal to the mast 104, and a pin (not shown). The frame 148, mast 104, boom 106, and other structural components of the apparatus 100 may be made of a suitable structural material, for example, steel.

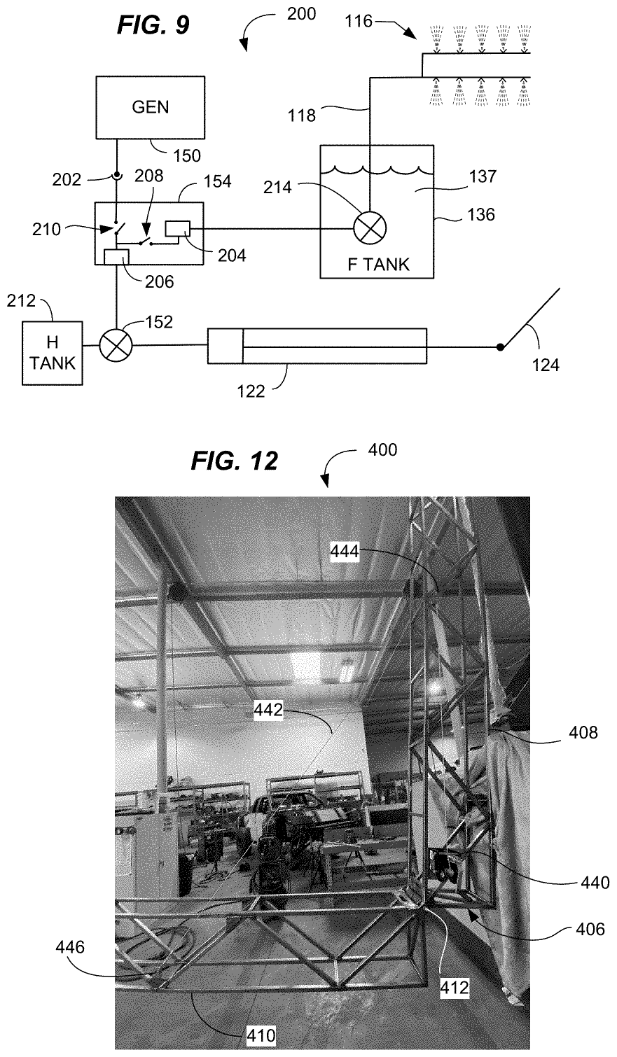

The apparatus 100 may further include a fluid container 136 for holding any suitable air quality control fluid 137 (see FIG. 9). The container 136 may be coupled to the base 102. Suitable air quality control fluids are known in the art, for example, water-based fluids including an enzyme for breaking apart odor molecules and/or perfumes for masking odors. A fluid pump 214 (FIG. 9) may be coupled to the container 136 and to an output fluid line 118 disposed for directing air quality control fluid 137 along the boom 106 to an array 116 of fluid dispensing nozzles 117 extending along a length of the boom. The fluid container 136 may be a plastic container mounted to the base. The array 116 of nozzles 117 is coupled to receive the air quality control fluid 137 from the output fluid line 118. The array 116 of fluid dispensing nozzles 117 may be oriented to emit the air quality control fluid downwards. Alternatively, the nozzles 117 may be oriented in any other orientation desired depending on the area to be covered, the contaminant to be controlled, and the mode of operation of the air quality control fluid. For example, a downward orientation may be preferred in applications where the fluid is to be dispersed over the ground, while a horizontal or upward orientation may be preferred where the control fluid is designed to work while airborne. The desired orientation may also depend on wind conditions. In some embodiments, the nozzles 117 may be designed to rotate in different orientations during operation, or to have an adjustable orientation when the apparatus is not operating to dispense fluid. In other embodiments, the nozzles 117 may be set at different orientations to disperse the fluid over a wider area. For example, a first third of the number of nozzles may be set to dispense fluid horizontally in a first direction, a second third set to dispense horizontally opposite to the first direction, and a last third to dispense perpendicularly to the horizontal nozzles, e.g., upwards or downwards.

The fluid line 118 may comprise branches as shown in FIG. 9, wherein one branch extends along each lower rail 111, 113 of the boom 106. The boom 106 may be, or may include, a truss including the lower rails 111, 113. The fluid line may include a slack portion as shown in FIG. 2 to facilitate folding of the boom 106. The dispensing nozzles 117 may be designed to emit fine droplets, for example, a mist.

The apparatus 100 may further include a piston-driven actuator, for example, a hydraulic cylinder 122, interposed between the boom 106 and at least one of the base 102 or the mast 104, configured for moving the boom 106 between the horizontally extended position and the folded position. The cylinder 122 may be arranged to drive a pivoting armature 120, comprising a pivoting double-ended clevis 120 connected to a rod 126, which is coupled to the boom 106 by a bracket 128. Extending the cylinder 122 causes the distal end of the pivoting clevis to rotate downward, lowering the boom 106. Other actuators and armatures may also be suitable for lowering and raising the boom.

The boom 106 may be divided into two or more segments 108, 110, for example, a proximal segment 108 coupled to a distal segment 110 by a hinge 112 enabling the boom to be folded when lowered. In an aspect, the hinge 112 may be spring-loaded to assist manual folding of the boom when lowered. In an alternative, or in addition, a power-assist mechanism may be provided to ease the manual effort required to fold or unfold the boom. An example of a power-assist mechanism 400 is described below in conjunction with FIG. 12.

Additionally, a distal end of the distal segment 110 may be equipped with wheels to facilitate lowering the boom. As the proximal segment 108 is lowered toward the mast 104, the wheels 114 on the distal segment 110 allow it to roll across the ground toward the base 102. When the proximal segment 108 is lowered near the base 102, a worker may with assistance from the spring-loaded hinge 112 and/or power-assist mechanism 400 lift the distal segment 110 against the proximal segment 108, as shown in FIGS. 3-4. Thus, the boom can be lowered and folded to facilitate relocating the apparatus 100 as the odor source moves. When folded, the two segments 108, 110 may be secured together by a safety clamp 140, or by several such clamps.

In some embodiments, the piston driven actuator 122 may be a hydraulic cylinder. Accordingly, the apparatus 100 may further include a hydraulic system comprising a fluid reservoir 212 and hydraulic pump 152 coupled to the hydraulic cylinder 122 (FIG. 9).

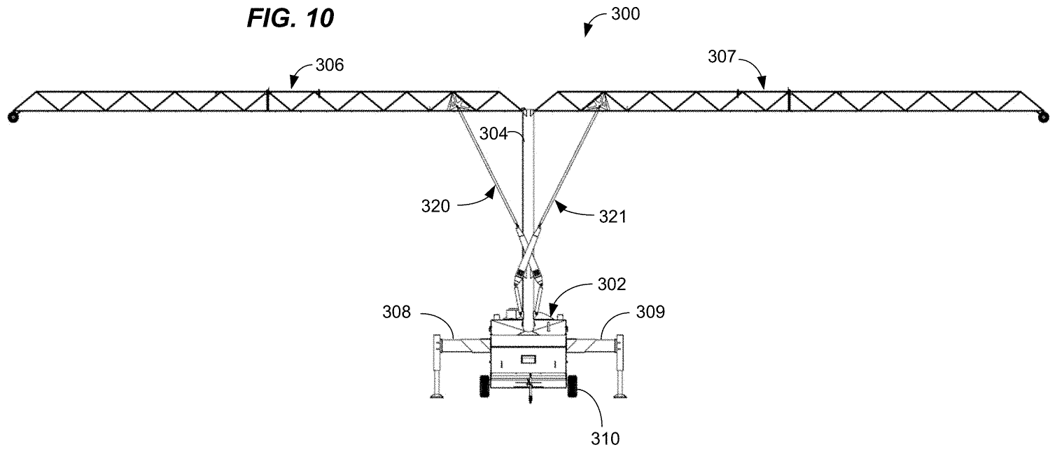

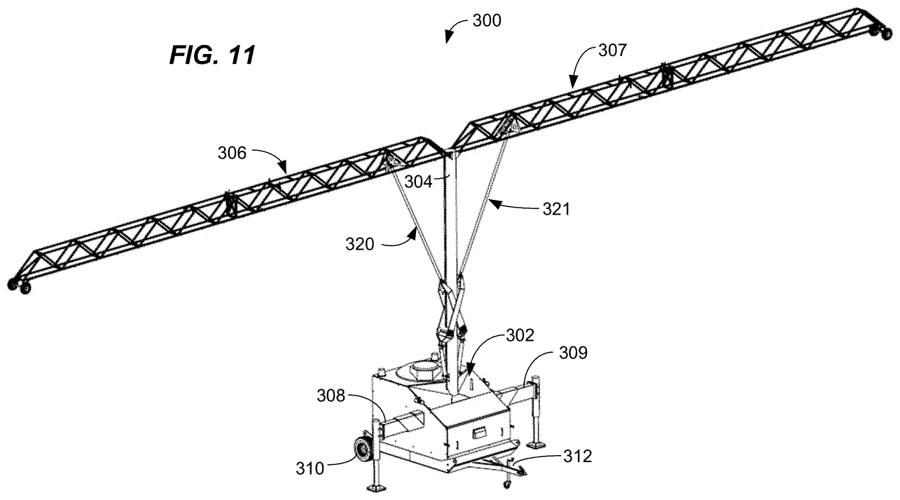

The apparatus may include at least one skid 130 supporting the base 102. For example, the skid 130 may be, or may include, a steel plate. The skid allows the apparatus 100 to be readily dragged into position when the boom 106 is lowered and folded. In the alternative to a skid, the base 106 may be configured as a trailer with wheels as shown in FIGS. 10-11.

A first horizontal dimension L1 of the apparatus 100, shown in FIG. 2, may be approximately 43.5 feet. A length of the base L2 may be approximately 6 feet. A first vertical dimension H1 of the apparatus 100 may be approximately 23.25 feet. A height of the base H2 may be approximately 5.3 feet. Referring to FIG. 3, a height H3 of the apparatus 100 when fully folded may be approximately 22.75 feet, and its length L3 may be approximately 9.5 feet. Referring to FIG. 4, an overall width W1 of the base may be approximately 9.25 feet and a width W2 of the base frame may be approximately 7.5 feet. All the foregoing dimensions are merely examples and may vary within functional limits.

Referring to FIGS. 5-8, the base 102 may comprise a frame 148 made of welded tubular steel covered by sheet metal panels 132 with an access door 134 protecting an interior of the base 102. The interior may thus be used to house electrical, fluid, and hydraulic components, for example a control panel 154, hydraulic pump 150 and portable electrical generator 152. The interior may also hold a container 135 for the air quality control fluid.

Referring to FIG. 9, the apparatus 100 may further include an electrical system 200 coupled to the fluid pump 214. The electrical system may include a control panel 154 coupled to a source of electric power (e.g., a portable generator 150) via a plug-and-socket port 202. The control panel may include one or more switches, for example a first switch 210 for controlling power from the power source 150 to a power conditioner 206 for the hydraulic pump 152, and a second switch 208 for controlling power to a power conditioner 204 for the fluid pump 214 that pumps the air quality control fluid 137. Additional switches (not shown) may be used to control power to other powered components of the apparatus 100, for example, to a power-assist mechanism 400 described in connection with FIG. 12 below.

Referring to FIGS. 10-11, in alternative embodiments, an apparatus 300 may include dual booms 306, 307, and dual armatures 320, 321 for raising and lowering respective ones of the booms. The booms 306, 307 may be mounted to a single mast 304 and driven as described for the single boom apparatus 100. In a separate aspect, alternative embodiments may provide the base 302 on a trailer with wheels 310 and a hitch 312, which may be stabilized when parked by outriggers 309, 310.

Referring to FIG. 12, a power-assist mechanism 400 may be coupled to any one of the booms 106, 306, or 307 to assist with folding or unfolding of the segments thereof. For example, a boom 406 having a first segment 408 proximal to a mast coupled to a distal segment 410 by a hinge 412 may include the power-assist mechanism 400. The mechanism 400 may include a cable 442 attached to a bracket 446 fixed to the distal segment, using any suitable fastener (e.g., bolt, clevis, hook, eyebolt, etc.). The mechanism 400 may further include a pully 444 fixed to the proximal segment 408. The cable 442 may be run from the bracket 446 through the pully 444 to a winch 440 attached to the proximal segment 408. The winch 440, which may be hand cranked or electrically powered, may be used to wind up the cable 442, drawing the bracket 446 and attached distal segment 410 of the boom 406 up to the pully 408, thereby pulling the two segments together into a fully folded position as shown in FIG. 3. The winch-assisted folding operation may begin after the boom 406 is lowered to the position shown in FIG. 12. Other power-assist mechanisms may also be suitable, for example, a hydraulic ram.

Unfolding the boom 406 is the reverse of folding. Any clamps securing the segments 408, 410 together are released and the winch 440 is unwound, causing the distal segment 410 to pivot downward around the hinge 412 to the position shown in FIG. 12. Once in this position, the winch can be fully unwound, and the proximal segment 408 raised using a lift 120 such as shown in connection with FIGS. 1-3 until the boom 406 is fully raised and extended like the boom 106 shown in FIG. 2.

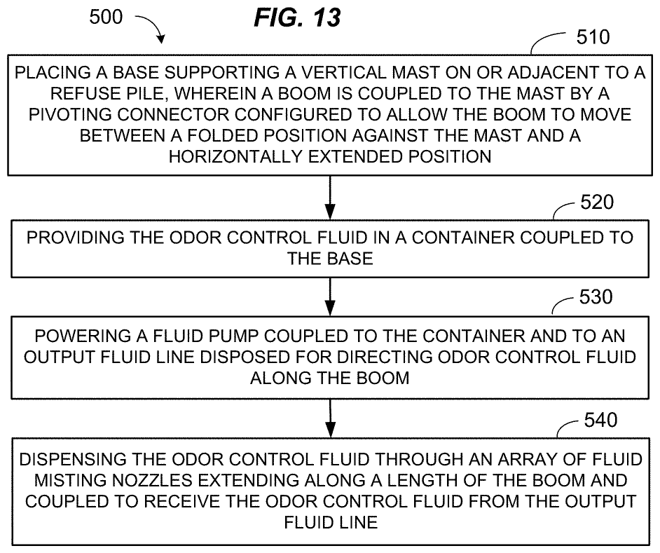

Referring to FIG. 13, a method 500 for dispensing an air quality control fluid over a refuse pile may include, at 510 placing an apparatus on or adjacent to a refuse pile, the apparatus including a base supporting a vertical mast, wherein a boom is coupled to the mast by a pivoting connector configured to allow the boom to move between a folded position against the mast and a horizontally extended position. The method 500 may further include, at 520, providing the air quality control fluid in a container coupled to the base. The method 500 may further include, at 530, powering a fluid pump coupled to the container and to an output fluid line disposed for directing air quality control fluid along the boom. The method 500 may further include, at 540 dispensing the air quality control fluid through an array of fluid dispensing nozzles extending along a length of the boom and coupled to receive the air quality control fluid from the output fluid line. In an aspect, the air quality control fluid may be, or may include, any one or more fluids selected from an odor control fluid, a dust control fluid, or a chemical contaminant control fluid.

Other aspects of the method 500 may include, for example folding segments of the boom to place the boom in the folded position, dragging the apparatus on a skid, and/or generating a mist of the air quality control fluid.

The previous description of the disclosed aspects is provided to enable any person skilled in the art to make or use the present disclosure. Various modifications to these aspects will be clear to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. An apparatus for dispensing an air quality control fluid over refuse, the apparatus comprising:
a base supporting a vertical mast;
a boom coupled to the mast by a pivoting connector configured to allow the boom to move between a folded position against the mast and a horizontally extended position;
a container for holding an air quality control fluid coupled to the base;
a fluid pump coupled to the container and to an output fluid line disposed for directing air quality control fluid along the boom; and
an array of fluid dispensing nozzles extending along a length of the boom and coupled to receive the air quality control fluid from the output fluid line.

2. The apparatus of claim 1, further comprising a piston-driven actuator interposed between the boom and at least one of the base or the mast, configured for moving the boom between the horizontally extended position and the folded position.

3. The apparatus of claim 2, further comprising a hinge that couples segments of the boom together, enabling the boom to be folded.

4. The apparatus of claim 3, wherein the hinge is spring-loaded to assist folding of the boom when lowered.

5. The apparatus of claim 2, wherein the piston driven actuator comprises a hydraulic cylinder.

6. The apparatus of claim 5, further comprising a hydraulic system coupled to the hydraulic cylinder.

7. The apparatus of claim 1, further comprising at least one skid supporting the base.

8. The apparatus of claim 7, wherein the skid comprises a steel plate.

9. The apparatus of claim 1, further comprising an electrical system coupled to the fluid pump.

10. The apparatus of claim 9, further comprising a control panel coupled to the electrical system.

11. The apparatus of claim 9, further comprising an electrical port for connecting a source of electric power to the electrical system.

12. The apparatus of claim 11, further comprising a portable generator coupled to the electrical system via the port.

13. The apparatus of claim 1, wherein the boom comprises a truss.

14. The apparatus of claim 1, wherein the fluid container comprises a plastic container mounted to the base.

15. The apparatus of claim 1, further comprising a power-assist mechanism coupled to segments of the boom and configured to supply power for folding the segments of the boom together.

16. A method for dispensing an air quality control fluid over a refuse pile, comprising:
placing an apparatus for dispensing an air quality control fluid on or adjacent to a refuse pile, wherein the apparatus comprises a base supporting a vertical mast, a boom is coupled to the mast by a pivoting connector configured to allow the boom to move between a folded position against the mast and a horizontally extended position;
providing the air quality control fluid in a container coupled to the base;
powering a fluid pump coupled to the container and to an output fluid line disposed for directing air quality control fluid along the boom; and
dispensing the air quality control fluid through an array of fluid dispensing nozzles extending along a length of the boom and coupled to receive the air quality control fluid from the output fluid line.

17. The method of claim 16, further comprising driving a piston-driven actuator interposed between the boom and at least one of the base or the mast to move the boom between the horizontally extended position and the folded position.

18. The method of claim 16, further comprising folding segments of the boom to place the boom in the folded position.

19. The method of claim 16, further comprising dragging the apparatus on a skid.

20. The method of claim 16, wherein the dispensing comprises generating a mist of the air quality control fluid.
